(19) United States
(12) Patent Application Publication
(10) Pub. No.: US 2024/0197516 A1
(43) Pub. Date: Jun. 20, 2024

(54) Wearable Support Structure For At Least Partly Relieving A Human Body During Leaning Or Bending Over

(71) Applicant: Laevo B.V., Rijswijk (NL)

(72) Inventors: Boudewijn Martin Wisse, Rijswijk (NL); Michael Martinus Hölscher, Rijswijk (NL); Sebastiaan Johannes Wagemaker, Rijswijk (NL)

(21) Appl. No.: 17/909,831

(22) PCT Filed: Mar. 10, 2021
(86) PCT No.: PCT/NL2021/050160

§ 371 Date: Sep. 7, 2022

(30) Foreign Application Priority Data
Mar. 18, 2020 (NL) 2025157

(51) Int. Cl. A61F 5/02; A61F 5/01

(52) U.S. Cl. CPC A61F 5/028; A61F 5/0102

(57) ABSTRACT

A wearable support structure includes a torso support, two elongated resilient stays connected to the torso support, a lumbar support, two thigh supports, two pivoting arrangements, and a movement arrangement. The torso support is arranged for bearing upon a chest and/or a back of the human body. The lumbar support is arranged to extend substantially alongside the human body and is in a first state when being unloaded, and is biased back to the first state when being into a second state corresponding with a leaning or bending position of the human body. The two pivoting arrangements each connect a respective elongated resilient stay to a respective thigh support and allow the respective resilient stay and thigh support to pivot about a main axis. The movement arrangement maintains the two resilient stays in the first state when the human body is in the upright position.

The present disclosure relates to a wearable support structure for at least partly relieving a human body during leaning or bending over, said wearable support structure comprising:
a torso support arranged for bearing upon a chest and/or a back of said human body;
two elongated resilient stays, connected to said torso support, arranged to extend substantially alongside said human body and arranged for being in a first state when being unloaded, and arranged to be biased back to said first state when being into a second state corresponding with a leaning or bending position of said human body;
a lumbar support arranged for bearing upon a lumbar area of said human body;
two thigh supports, each arranged for bearing upon a respective thigh area of said human body, preferably a front side of a respective thigh area of said human body;
two pivoting arrangements, each connecting a respective elongated resilient stay of said two elongated resilient stays to a respective thigh support of said two thigh supports and allowing said respective elongated resilient stay and said respective thigh support to pivot about a main axis.

NL2014451 discloses a wearable support structure. One of the downsides of the wearable support structure disclosed by NL2014451 is that the known wearable support structure is relatively difficult to adapt to different levels of support of the human body. Using the known wearable support structures a human body is also biased back, by the two elongated resilient stays, to an upright position in situations wherein such biasing back is not desired, or at least desired to a different extent. Examples of such situations may occur when the wearer of the support structure is walking. During walking the biasing back to the upright position may be perceived as rather uncomfortable in that the chest support keeps pressing against the human body at a relative large biasing force. As such, a user wearing the known wearable support structure may feel hindered or restricted to walk while wearing the known wearable support structure.

It is therefore an object of the present invention to provide for a wearable support structure that is relative comfortable for the user during walking.

The object is achieved, in that the wearable support structure according to the present disclosure comprises a movement arrangement arranged for maintaining said two elongated resilient stays in said first state when said human body is in said upright position. By providing the movement arrangement it is avoided that the two elongated resilient stays are in said second state when said human body is in an upright position. The movement arrangement realises that the two elongated resilient stays are in said second position only when said human body is leaning or bending. This allows the user to move, for instance during walking, in the upright position without experiencing a biasing force, or at least experiencing only a relative small biasing force.

An additional advantage of providing the movement arrangement is that there is no need for a user of the wearable support structure according to the present disclosure to actively bring the wearable support structure in a condition wherein the user may move freely without experiencing the biasing force.

The movement arrangement accommodates for out-of-phase movements of the user of the wearable support structure in the sagittal plane of the human body.

Within the context of the present disclosure, the chest and the back of the human body referred to in relation to the torso support being arranged for bearing upon the chest and/or the back of said human body are to be understood as a part of the human body between the shoulders and the lumbar area of the human body. In other words, when a user is wearing the wearable support structure the torso support bears upon a part of the human body that is between the shoulders and the lumbar area of the user.

Within the context of the present disclosure, the lumbar area is to be understood as a part of the human body between the upper side of thighs and lower part of the back of the human body. The lumbar area may for instance refers to the hip, buttocks and an area of the back of the human body in proximity of the lower spine.

Preferably, said two thigh supports are arranged for being in a first state when being unloaded, and arranged to be biased back to said first state when being into a second state corresponding with a leaning or bending position of said human body. This is beneficial for providing a wearable support structure that is relative comfortable for the user during walking.

In this regard, it is advantageous if said movement arrangement is arranged for maintaining said two thigh supports in said first state when said human body is in said upright position. This is beneficial for providing a wearable support structure that is relative comfortable for the user during walking.

In an embodiment of the wearable support structure according to the present disclosure, said movement arrangement is arranged for maintaining a pivot angle about said main axis of said respective elongated resilient stay of said two elongated resilient stays substantially equal, preferably equal, to a pivot angle about said main axis of said respective thigh support of said two thigh supports when said human body is in said upright position. In other words, the movement arrangement is arrange for realising that the elongated resilient stay and the thigh support at a side of the human body pivot over the substantially the same angle about the main axis of the pivoting structure connecting the elongated resilient stay and the thigh support when the user of the wearable support structure is walking and thereby allowing the user to walk without experiencing a biasing force, or at least experiencing only a relative small biasing force.

Preferably, said movement arrangement comprises a first pivot structure, wherein said torso support is pivotally connected, via said first pivot structure to said two elongated resilient stays. Providing the first pivot structure is beneficial for allowing a movement between the torso support and the two elongated resilient stays. Such a movement between the torso support and the two elongated resilient states allows for the torso of the human body to move relative to the two elongated resilient stays while maintaining the two elongated resilient stays in the first state. This is beneficial for providing a wearable support structure that is relative comfortable for the user during walking.

In this regard, it is beneficial if said first pivot structure is arranged for pivoting said torso support relative to said two elongated resilient stays about a first pivot axis and a second pivot axis, wherein said first pivot axis is at right angles to said second pivot axis. This is beneficial for providing a wearable support structure that is relative comfortable for the user during walking.

Preferably, said first pivot axis, when said torso support is bearing upon said chest and/or said back of said human body, extends in a direction comprising a component in a vertical direction when said human body is in said upright position. In other words, the first pivot axis comprises a component in a direction that extends in a longitudinal direction of the human body. Within the context of the present disclosure, the longitudinal direction of the human body is to be understood as a direction perpendicular to the ground surface when the human body is in the upright position.

In this regard, it is beneficial if said first pivot structure is further arranged for pivoting said torso support relative to said two elongated resilient stays about a third pivot axis, wherein said third pivot axis is at right angles to said first pivot axis and said second pivot axis. Allowing said elongated resilient stays to pivot about the third pivot axis relative to the torso support is beneficial for reducing wringing forces that may be present in the wearable support structure during use of the wearable support structure. This is beneficial for providing a wearable support structure that is relative comfortable for the user during walking.

Preferably, said first pivot structure comprises an omnidirectional pivot coupling, preferably a ball-joint coupling, for allowing omnidirectional pivoting of said torso support relative to said two elongated resilient stays. This is beneficial for providing a wearable support structure that is relative comfortable for the user during walking.

In this regard, it is beneficial if said ball-joint coupling is arranged for pivoting of said torso support relative to said two elongated resilient stays about said first, second and/or third pivot axis.

In an embodiment of the wearable support structure according to the present disclosure, said omnidirectional pivot coupling comprises a flexible element for allowing said omnidirectional pivoting of said torso support relative to said two elongated resilient stays.

It is beneficial if said first pivot structure comprises a translation arrangement arranged for allowing said torso support to translate relative to said two elongated resilient stays. A translation arrangement is beneficial for avoiding, or at least significantly reducing, transfer of a shear force between the torso support and the human body. Such shear forces may be induced by tilt and rotation of the hip during gait of the human body. Allowing the torso support to translate relative to the two elongated resilient stays allows the torso support to bear upon the chest and/or back of the human body while avoiding, or at least significantly reducing, a movement of the torso support relative to the chest and/or back of the human body during gait of the user. This is beneficial for providing a wearable support structure that is relative comfortable for the user during walking.

In this regard, it is advantageous if said translation arrangement comprises a further omnidirectional pivot coupling, preferably a further ball-joint, for allowing said torso support to translate relative to said two elongated resilient stays, preferably for allowing said torso support to translate over a predetermined distance relative to said two elongated resilient stays. Providing a further ball-joint is beneficial for realising a wearable support structure wherein said torso support, when said two elongated resilient stays are in said second state, is biased back into a first position relative to said two elongated resilient stays. This is beneficial for providing a wearable support structure that is relative comfortable for the user during walking while realising a biasing back of the two elongated resilient stays that is relative comfortable for the user.

Preferably, said translation arrangement is arranged for translating said torso support in two perpendicular directions in a flat virtual plane relative to said two elongated resilient stays. This is beneficial for avoiding, or at least significantly reducing, transfer of a shear force between the torso support and the human body acting in the flat virtual plane. This is beneficial for providing a wearable support structure that is relative comfortable for the user during walking.

It is advantageous if said movement arrangement is arranged for allowing said two elongated resilient stays to pivot independent from each other relative to said torso support. This is beneficial for providing a wearable support structure that is relative comfortable for the user during walking.

Preferably, said movement arrangement is arranged for allowing said two elongated resilient stays to pivot independent from each other relative to said torso support about said second pivot axis.

Preferably, said movement arrangement further comprises a second pivot structure for allowing each of said elongated resilient stays to pivot about a fourth pivot axis relative to said pivoting arrangement connecting said elongated resilient stay, via said pivoting arrangement, to said thigh support, wherein said fourth pivot axis extends along said elongated resilient stay.

In this regard, it is beneficial if said fourth pivot axis is perpendicular to said main axis.

Preferably, the first pivot axis is substantially parallel, preferably parallel, to the fourth pivot axis. This is beneficial for allowing for a relative large stroke of the legs of the user in the upright position while maintaining the two elongated resilient stays in the first state. This is beneficial for providing a wearable support structure that is relative comfortable for the user during walking.

Preferably, said fourth pivot axis is provided outside said first pivot structure. This is beneficial for allowing for a relative large stroke of the legs of the user in the upright position while maintaining the two elongated resilient stays in the first state. This is beneficial for providing a wearable support structure that is relative comfortable for the user during walking.

In an embodiment of the wearable support structure according to the present disclosure, said main axes of said pivoting arrangements are concentric. This is beneficial for allowing for a relative large stroke of the legs of the user in the upright position while maintaining the two elongated resilient stays in the first state. This is beneficial for providing a wearable support structure that is relative comfortable for the user during walking.

It is beneficial if said wearable support structure comprises a third pivot structure, wherein said lumbar support is pivotally connected, via said third pivot structure, to said two pivoting arrangements. This is beneficial for providing a wearable support structure that is relative comfortable for the user during walking.

In this regard, it is beneficial if said third pivot structure is arranged for pivoting said lumbar support relative to said two pivoting arrangements about a fifth pivot axis. This is beneficial for providing a wearable support structure that is relative comfortable to wear during walking.

Preferably, said fifth pivot axis is concentric with said main axes. This is beneficial for providing a wearable support structure that is relative comfortable for the user during walking.

Preferably, said second pivot structure is arranged for pivoting said elongated resilient stays about a sixth pivot axis relative to said pivoting arrangements. Allowing said elongated resilient stays to pivot about a sixth pivot axis relative to said pivoting arrangements is beneficial for reducing wringing forces that may be present in the wearable support structure during use of the wearable support structure. This is beneficial for providing a wearable support structure that is relative comfortable for the user during walking.

Preferably, said sixth pivot axis is substantially at right angles, preferably at right angles, to the main axis and the fourth axis.

In an embodiment of the wearable support structure according to the present disclosure, said two pivoting arrangements are arranged for allowing said respective elongated resilient stay, connected to a respective pivoting arrangement of said two pivoting arrangements, to pivot about said main axis relative to said respective thigh support, connected to said respective pivoting arrangement of said two pivoting arrangements, wherein each of said two pivoting arrangements is arranged to provide a biasing force biasing said respective elongated resilient stay into a predetermined angle relative to said respective thigh support, wherein said movement arrangement is arranged for maintaining said respective elongated resilient stay into said predetermined angle relative to said respective thigh support and maintaining said two elongated resilient stays in said first state when said human body is in said upright position.

Preferably, said predetermined angle is in the range of 150 to 180 degrees.

In an embodiment of the wearable support structure according to the present disclosure, said wearable support structure comprises a fourth pivot structure for pivoting said thigh supports relative to said pivoting arrangements about a seventh pivot axis for allowing a user of said wearable support structure to move said thigh areas away from each other in a direction along said main axis.

The present invention will now be explained by means of a description of preferred embodiments of a wearable support structure according to the present disclosure, in which reference is made to the following schematic figures, in which:

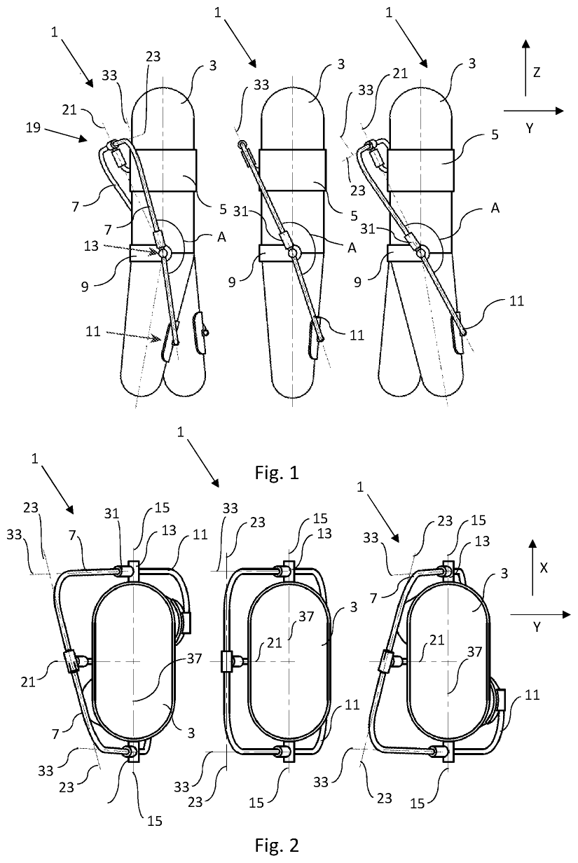

FIG. 1: a side view of human bodies wearing a wearable support structure according to the present disclosure are shown;

FIG. 2: a top view of the human bodies from FIG. 1 are shown;

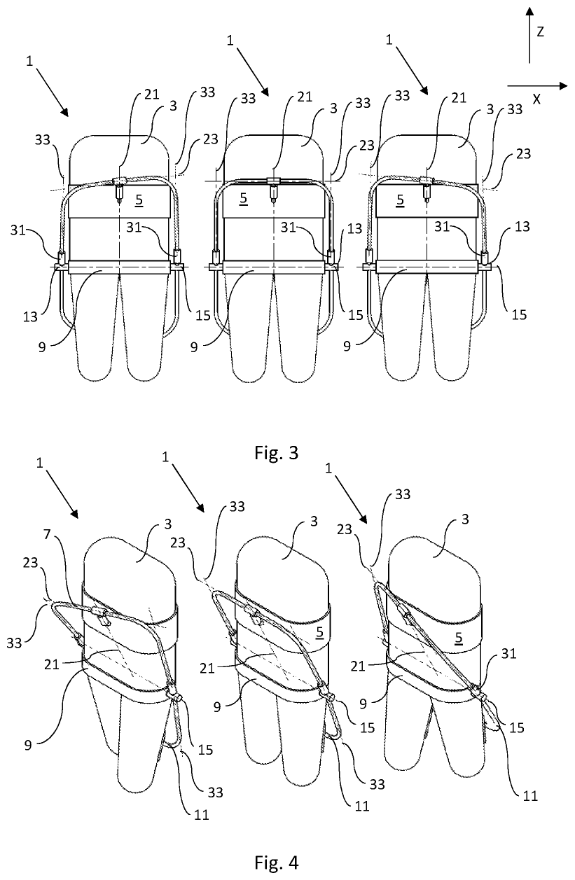

FIG. 3: a back view of the human bodies from FIG. 1 are shown;

FIG. 4: an isometric view of the human bodies from FIG. 1 are shown;

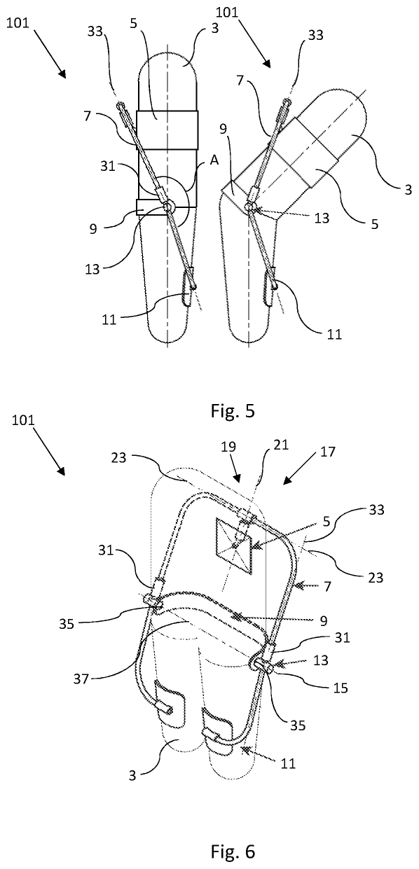

FIG. 5: a side view of human bodies wearing a wearable support structure according to the present disclosure are shown;

FIG. 6: an isometric view of a human body from FIG. 6 is shown;

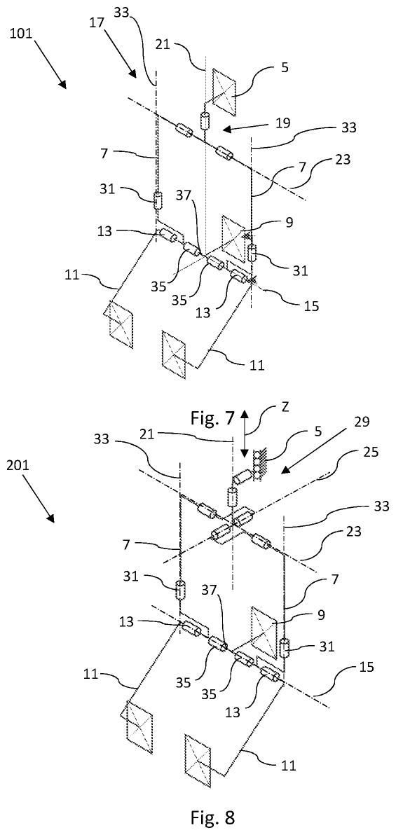

FIG. 7: a wearable support structure according to the present disclosure is shown;

FIG. 8: a further wearable support structure according to the present disclosure is shown;

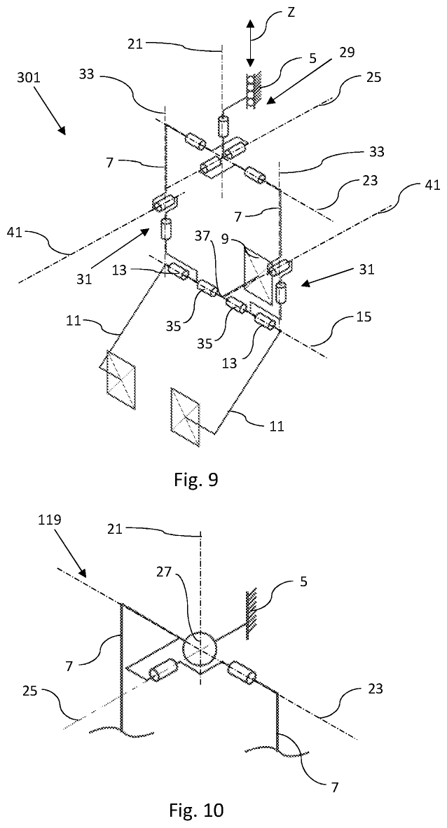

FIG. 9: another wearable support structure according to the present disclosure is shown;

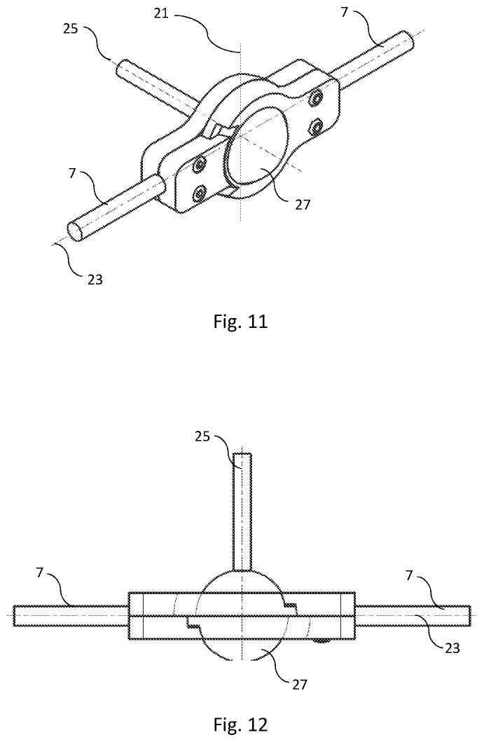

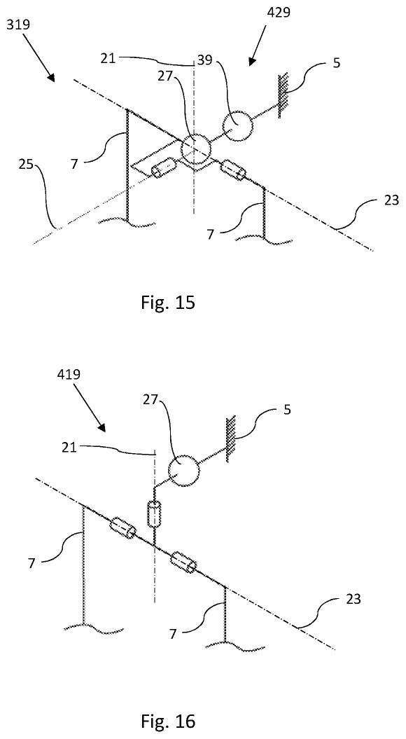

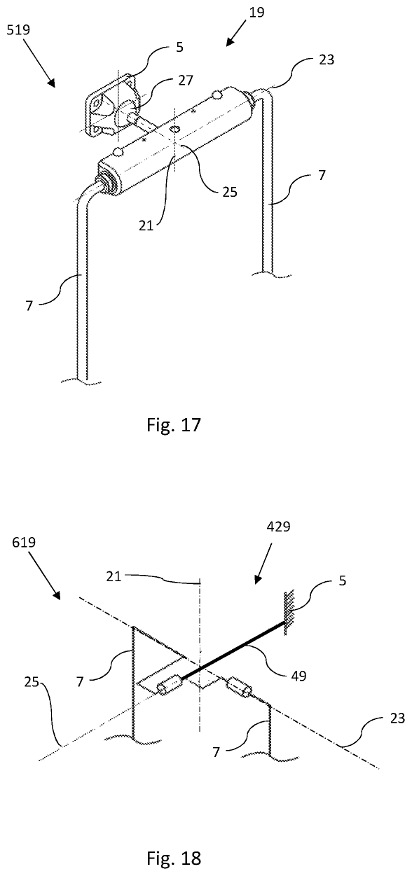

FIGS. 10-18: elements of a wearable support structure according to the present disclosure are shown.

Wearable support structure 1 comprises a torso support 5 arranged for bearing upon a chest and a back of the human body 3. The torso support 5 comprises a belt that may be provided at the location of the chest around the torso of the human body 3. The torso support 5 attaches the wearable support structure 1 in a relative firm manner to the human body 3. Two elongated resilient stays 7 are provided and connected to said torso support 5. The two elongated resilient stays 7 extend substantially alongside the human body 3 and are arranged for being in a first state when being unloaded, and arranged to be biased back to said first state when being into a second state due to leaning or bending of the human body 3. The stays 7 may be substantially strip shaped, having a three-dimensional curved shape, the strip having for example a flat, curved, triangular or multi angular, round or oval cross section, which may be constant over the length of the stay or can vary along its length.

The wearable support structure 1 further comprises a lumbar support 9. The lumbar support 9 is arranged for bearing upon a lumbar area of said human body 3. At a lower end of the wearable support structure 1, the wearable support structure 1 is provided with two thigh supports 11. The thigh supports 11 are each arranged for bearing upon a front side of a respective thigh area of the human body 3. The thigh supports 11 may also be arranged for being in a first state when being unloaded, and arranged to be biased back to the first state when being into a second state due to leaning or bending of the human body 3. During leaning or bending the thigh supports 11 may press firmly against the thigh areas of the user.

A respective elongated resilient stay 7 of the two elongated resilient stays 7 and a respective thigh support 11 of the two thigh supports 11 are connected to a pivoting arrangement 13 of the wearable support structure 1. The pivoting arrangement 13 allows for pivoting the respective elongated resilient stay 7 and the respective thigh support 11 about a main axis 15. The main axes 15 of the pivoting arrangements 13 are concentric and preferably substantially parallel to a frontal axis of the human body 3 and substantially parallel to the axis over which the human body 3 bends forward. The main axis 15 may coincide with an axis through hip joints of the human body 3.

The lumbar support 9 is pivotally connected to the two pivoting arrangements 13 for pivoting, via a third pivot structure 35 of the wearable support structure 1, the lumbar support 9 about a fifth pivot axis 37. The fifth pivot axis 37 is concentric with the main axes 15 of the pivoting arrangements 13. The lumbar support 9 may be formed as a flexible belt that, at least during use of the wearable support structure 1, presses against the lumbar area of the user.

The wearable support structure 1 according to the present disclosure comprises a movement arrangement 17. The movement arrangement 17 is arranged for maintaining the two elongated resilient stays 7 and the two thigh supports 11 in their first states when the user of the wearable support structure 1 is walking. The movement arrangement 17 comprises a first pivot structure 19 and a second pivot structure 31.

The first pivot structure 19 connects the two elongated resilient stays 7 to the torso support 5. The torso support 5 may pivot, via the first pivot structure 19, relative to the two elongated resilient stays 7 about a first pivot axis 21 and a second pivot axis 23. The first pivot axis 21 is at right angles to the second pivot axis 23. In addition, the first pivot axis 21 extends in a direction comprising a component in a vertical direction Z when the human body 3 is in the upright position shown in FIGS. 1-4. The first pivot structure 19 is arranged for allowing the two elongated resilient stays 7 to pivot independent about the second pivot axis 23 from each other relative to the torso support 5.

The second pivot structure 31 connects the elongated resilient stays 7 to the pivoting arrangements 13 and allows each of the elongated resilient stays 7 to pivot about a fourth pivot axis 33 relative to the pivoting arrangement 13. The fourth pivot axis 33 extends along the elongated resilient stay 7 and is perpendicular to the main axis 15.

Wearable support structure 101 differs mainly from wearable support structure 1 in that the two pivoting arrangements 13 are arranged for allowing the respective elongated resilient stay 7, connected to a respective pivoting arrangement 13 of the two pivoting arrangements 13, to pivot about the main axis 15 relative to the respective thigh support 11, connected to the respective pivoting arrangement 13 of the two pivoting arrangements 13. The pivoting arrangements 13 of the wearable support structure 101 are each arranged to provide a biasing force biasing the respective elongated resilient stay 7 into a predetermined angle A relative to the respective thigh support 11. In addition, the movement arrangement 17 of wearable support structure 101 is arranged for maintaining the respective elongated resilient stay 7 into the predetermined angle A relative to the respective thigh support 11 and maintaining the two elongated resilient stays 7 in the first state when the human body 3 is in the upright position and moving such as walking. Elements of wearable support structure 101 similar to elements of wearable support structure 1 are indicated by the same reference numbers.

Wearable support structure 201 differs mainly from wearable support structure 101 in that the first pivot structure of wearable support structure 201 is further arranged for pivoting the torso support 5 relative to the two elongated resilient stays 7 about a third pivot axis 25. The third pivot axis 25 is at right angles to the first pivot axis 21 and the second pivot axis 23. In addition, the first pivot structure of wearable support structure 201 comprises a translation arrangement 29 arranged for allowing the torso support 5 to translate relative to the two elongated resilient stays 7 in a direction having a component in the vertical direction Z. Elements of wearable support structure 201 similar to elements of wearable support structure 1 and/or 101 are indicated by the same reference numbers.

Wearable support structure 301 differs mainly from wearable support structure 201 in that the second pivot structure 31 of the wearable support structure 301 is arranged for pivoting the elongated resilient stays 7 about a sixth pivot axis 41 relative to the pivoting arrangement 13. The sixth pivot axis 41 is at substantially right angles relative to the main axis 15 and the fourth axis 33. Elements of wearable support structure 301 similar to elements of wearable support structure 1, 101 and/or 201 are indicated by the same reference numbers.

The first pivot structure 119 shown in FIG. 10 may be part of wearable support structures 1, 101, 201 or 301 and comprises a ball-joint coupling 27. The ball-joint coupling 27 allows for an omnidirectional pivoting of the torso support 5 relative to said two elongated resilient stays 7. The two elongated resilient stays 7 are mutually connected such that the two elongated resilient stays 7 may pivot relative to each other about the second pivot axis 23 and the third pivot axis 25. Elements of first pivot structure 119 that are similar to elements of first pivot structure 19 are indicated by the same reference numbers.

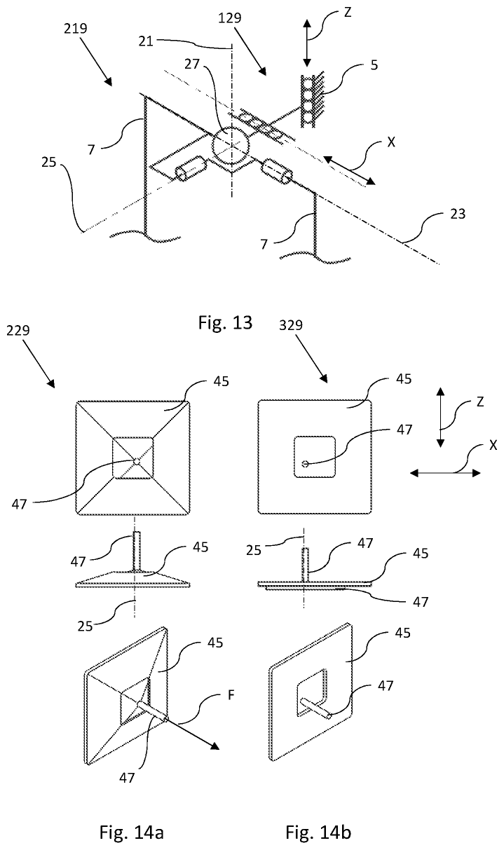

The first pivot structure 219 shown in FIG. 13 may be part of wearable support structures 1, 101, 201 or 301 and comprises a ball-joint coupling 27 and a translation arrangement 129. The translation arrangement 129 is arranged for allowing the torso support 5 to translate relative to the two elongated resilient stays 7 in directions X and Z. Elements of first pivot structure 219 that are similar to elements of first pivot structure 19 and/or 119 are indicated by the same reference numbers.

The translation arrangement 229 differs mainly from translation arrangement 129 in that translation arrangement 229 comprises a housing 45 and a translation part 47. The housing 45 is arranged for coupling the first pivot structure to the torso support 5. The translation part 47 may be displaced in the directions X and Z relative to the housing 45 for allowing the torso support 5 to translate relative to the two elongated resilient stays 7. The translation part 47 is received in a receiving space (not shown) of the housing 45. The receiving space and the translation part 47 are formed such that the translation part 47 is in a predetermine position when exerting a force F on the translation part 47 in direction Y, wherein direction Y is perpendicular to direction X and direction Z. Preferably, the predetermined position of the translation part 47 corresponds with a centre of the housing 45 in a virtual plane extending in the directions X and Z. Elements of translation arrangement 229 that are similar to elements of translation arrangement 29 and/or 129 are indicated by the same reference numbers.

The translation arrangement 329 differs mainly from translation arrangement 229 in that the translation arrangement 329 is arranged for translating the torso support 5 in two perpendicular directions X and Z in a flat virtual plane relative to the two elongated resilient stays 7. Elements of translation arrangement 329 that are similar to elements of translation arrangement 29, 129 and/or 229 are indicated by the same reference numbers.

The first pivot structure 319 may be part of wearable support structures 1, 101, 201 or 301. The first pivot structure 319 differs mainly from first pivot structure 119 in that the first pivot structure 319 comprises a further ball-joint coupling 39. The ball-joint coupling 27 is connected to the torso support 5 via the further ball-joint coupling 39. The translation arrangement 429 of first pivot structure 319 comprises the ball-joint coupling 27 and the further ball-joint coupling 39. Elements of first pivot structure 319 that are similar to elements of first pivot structure 19, 119 and/or 219 are indicated by the same reference numbers. The first pivot structure 419 may be part of wearable support structures 1, 101, 201 or 301. The first pivot structure 419 differs mainly from first pivot structure 19 in that the first pivot structure 419 comprises a ball-joint coupling 27. Elements of first pivot structure 419 that are similar to elements of first pivot structure 19, 119, 219 and/or 319 are indicated by the same reference numbers.

The first pivot structure 519 may be part of wearable support structures 1, 101, 201 or 301. The first pivot structure 519 differs mainly from first pivot structure 19 in that the first pivot structure 519 comprises a ball-joint coupling 27. Elements of first pivot structure 519 that are similar to elements of first pivot structure 19, 119, 219, 319 and/or 419 are indicated by the same reference numbers.

The first pivot structure 619 may be part of wearable support structures 1, 101, 201 or 301. The first pivot structure 619 differs mainly from first pivot structure 319 in that the first pivot structure 619 comprises a flexible element 49, such as a cable or a wire, wherein the omnidirectional pivot coupling and the further omnidirectional pivot coupling are formed by the flexible element 49. Elements of first pivot structure 619 that are similar to elements of first pivot structure 19, 119, 219, 319, 419 and/or 519 are indicated by the same reference numbers.

1. Wearable support structure (1, 101, 201, 301) for at least partly relieving a human body (3) during leaning or bending over, said wearable support structure (1, 101, 201, 301) comprising:
a torso support (5) arranged for bearing upon a chest and/or a back of said human body (3);
two elongated resilient stays (7), connected to said torso support (5), arranged to extend substantially alongside said human body (3) and arranged for being in a first state when being unloaded, and arranged to be biased back to said first state when being into a second state corresponding with a leaning or bending position of said human body (3);
a lumbar support (9) arranged for bearing upon a lumbar area of said human body (3);
two thigh supports (11), each arranged for bearing upon a respective thigh area of said human body (3), arranged for being in a first state when being unloaded, and arranged to be biased back to said first state when being into a second state corresponding with a leaning or bending position of said human body (3);
two pivoting arrangements (13), each connecting a respective elongated resilient stay (7) of said two elongated resilient stays (7) to a respective thigh support (11) of said two thigh supports (11) and allowing said respective elongated resilient stay (7) and said respective thigh support (11) to pivot about a main axis (15),
wherein said wearable support structure (1, 101, 201, 301) comprises a movement arrangement (17) arranged for maintaining said two elongated resilient stays (7) and said two thigh supports (11) in said first states when said human body (3) is in an upright position and arranged for maintaining a pivot angle about said main axis (15) of said respective elongated resilient stay (7) of said two elongated resilient stays (7) substantially equal to a pivot angle about said main axis (15) of said respective thigh support (11) of said two thigh supports (11) when said human body (3) is in said upright position.

2. The wearable support structure (1, 101, 201, 301) according to claim 1, wherein said movement arrangement (17) comprises a first pivot structure (19, 119, 219, 319, 419, 519), wherein said torso support (5) is pivotally connected, via said first pivot structure (19, 119, 219, 319, 419, 519) to said two elongated resilient stays (7).

3. The wearable support structure (1, 101, 201, 301) according to claim 2, wherein said first pivot structure (19, 119, 219, 319, 419, 519) is arranged for pivoting said torso support (5) relative to said two elongated resilient stays (7) about a first pivot axis (21) and a second pivot axis (23), wherein said first pivot axis (21) is at right angles to said second pivot axis (23) and wherein said first pivot axis (21), when said torso support (5) is bearing upon said chest and/or said back of said human body (3), extends in a direction comprising a component in a vertical direction when said human body (3) is in said upright position.

4. The wearable support structure (201, 301) according to claim 3, wherein said first pivot structure (19, 119, 219, 319, 419, 519) is further arranged for pivoting said torso support (5) relative to said two elongated resilient stays (7) about a third pivot axis (25), wherein said third pivot axis (25) is at right angles to said first pivot axis (21) and said second pivot axis (23).

5. The wearable support structure according to claim 2, wherein said first pivot structure (119, 219, 319, 419, 519) comprises an omnidirectional pivot coupling for allowing omnidirectional pivoting of said torso support (5) relative to said two elongated resilient stays (7).

6. The wearable support structure (201, 301) according to claim 2, wherein said first pivot structure (219, 319) comprises a translation arrangement (29, 129, 229, 329, 429) arranged for allowing said torso support (5) to translate relative to said two elongated resilient stays (7).

7. The wearable support structure according to claim 5, wherein said translation arrangement (429) comprises a further omnidirectional pivot coupling for allowing said torso support (5) to translate relative to said two elongated resilient stays (7).

8. The wearable support structure according to claim 6, wherein said translation arrangement (329) is arranged for translating said torso support (5) in two perpendicular directions (X, Z) in a flat virtual plane relative to said two elongated resilient stays (7).

9. The wearable support structure (1, 101, 201, 301) according to claim 2, wherein said movement arrangement (17) is arranged for allowing said two elongated resilient stays (7) to pivot independent from each other relative to said torso support (5).

10. The wearable support structure (1, 101, 201, 301) according to claim 2, wherein said movement arrangement (17) further comprises a second pivot structure (31) for allowing each of said elongated resilient stays (7) to pivot about a fourth pivot axis (33) relative to said pivoting arrangement (13) connecting said elongated resilient stay (7), via said pivoting arrangement (13), to said thigh support (11), wherein said fourth pivot axis (33) extends along said elongated resilient stay (7).

11. The wearable support structure (1, 101, 201, 301) according to claim 10, wherein said fourth pivot axis (33) is perpendicular to said main axis (15).

12. The wearable support structure (1, 101, 201, 301) according to claim 1, wherein said main axes (15) of said pivoting arrangements (13) are concentric.

13. The wearable support structure (1, 101, 201, 301) according to claim 1, wherein said wearable support structure (1, 101, 201, 301) comprises a third pivot structure (35), wherein said lumbar support (9) is pivotally connected, via said third pivot structure (35), to said two pivoting arrangements (13).

14. The wearable support structure (1, 101, 201, 301) according to claim 13, wherein said third pivot structure (35) is arranged for pivoting said lumbar support (9) relative to said two pivoting arrangements (13) about a fifth pivot axis (37).

15. The wearable support structure (101, 201, 301) according to claim 1, wherein said two pivoting arrangements (13) are arranged for allowing said respective elongated resilient stay (7), connected to a respective pivoting arrangement (13) of said two pivoting arrangements (13), to pivot about said main axis (15) relative to said respective thigh support (11), connected to said respective pivoting arrangement (13) of said two pivoting arrangements (13), wherein each of said two pivoting arrangements (13) is arranged to provide a biasing force biasing said respective elongated resilient stay (7) into a predetermined angle (A) relative to said respective thigh support (11), wherein said movement arrangement (17) is arranged for maintaining said respective elongated resilient stay (7) into said predetermined angle (A) relative to said respective thigh support (11) and maintaining said two elongated resilient stays (7) in said first state when said human body (3) is in said upright position.

16. The wearable support structure according to claim 5, wherein said omnidirectional pivot coupling is a ball-joint coupling (27).

17. The wearable support structure according to claim 7, wherein said further omnidirectional pivot coupling is a further ball-joint (39).

18. The wearable support structure according to claim 7, wherein said further omnidirectional pivot coupling allows said torso support (5) to translate over a predetermined distance relative to said two elongated resilient stays (7).

19. The wearable support structure (1, 101, 201, 301) according to claim 14, wherein said fifth pivot axis (37) is concentric with said main axes (15).
